(19) United States
(12) Patent Application Publication
(10) Pub. No.: US 2025/0360318 A1
(43) Pub. Date: Nov. 27, 2025

(54) METHOD AND APPARATUS FOR NEUROSTIMULATION WITH DISCRETE DYNAMIC PATTERNS

(71) Applicant: Boston Scientific Neuromodulation Corporation, Valencia, CA (US)

(72) Inventors: Satya Venkata Sandeep Avvaru, Houston, TX (US); Tianhe Zhang, Studio City, CA (US)

(21) Appl. No.: 19/202,355

(22) Filed: May 8, 2025

Related U.S. Application Data
(60) Provisional application No. 63/650,628, filed on May 22, 2024.

(51) Int. Cl. A61N 1/36; A61N 1/372

(52) U.S. Cl. CPC A61N 1/3615; A61N 1/37247

(57) ABSTRACT

A system for delivering neurostimulation may include a programming control circuit configured to generate information for programming a stimulation device to control the delivery of the neurostimulation according to a neurostimulation program including a discrete dynamic pattern defined by stimulation parameters including at least one time-varying stimulation parameter. The system may also include a stimulation programming circuit configured to determine the neurostimulation program and including a dynamic pattern composer configured to determine the discrete dynamic pattern. The dynamic pattern composer may include a modulation function generator and a parameter modulator. The modulation function generator may be configured to receive discretization information and to determine a discretized modulation function using the received discretization information. The parameter modulator may be configured to select a stimulation parameter and to produce the at least one time-varying stimulation parameter by modulating the selected parameter using the discretized modulation function.

### CLAIM OF PRIORITY

This application claims the benefit of U.S. Provisional Application No. 63/650,628, filed on May 22, 2024, which is hereby incorporated by reference in its entirety.

### TECHNICAL FIELD

This document relates generally to neurostimulation and more particularly to a system and method for programming neurostimulation with discrete dynamic patterns each being a pattern of neurostimulation pulses defined using at least one stimulation parameter that is modulated by a discretized modulation function.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device is used to program the implantable neurostimulator with stimulation parameters controlling the delivery of the neurostimulation energy.

In one example, the neurostimulation energy is delivered to a patient in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Neurostimulation controlled using a pattern of neurostimulation pulses defined by constant stimulation parameters has been applied to effectively treat various disorders, while use of time-varying stimulation parameters can better resemble natural neural activities and hence have the potential of providing for better therapeutic effectiveness, when the stimulation parameters are properly programmed to utilize the beneficial effects of neurostimulation using one or more time-varying stimulation parameters.

### SUMMARY

In Example 1, a system for delivering neurostimulation from a stimulation device to a patient is provided. The system may include a programming control circuit and a stimulation programming circuit. The programming control circuit may be configured to generate information for programming the stimulation device to control the delivery of the neurostimulation according to a neurostimulation program including one or more dynamic stimulation patterns each defined by stimulation parameters including at least one time-varying stimulation parameter. The stimulation programming circuit may be configured to determine the neurostimulation program. The stimulation programming circuit may include a dynamic pattern composer configured to determine a discrete dynamic pattern of the one or more dynamic stimulation patterns. The dynamic pattern composer may include a modulation function generator and a parameter modulator. The modulation function generator may be configured to receive discretization information and to determine a discretized modulation function using the received discretization information. The discretized modulation function is a discretely time-varying function. The parameter modulator may be configured to select a parameter from the stimulation parameters defining the discrete dynamic pattern and to produce the at least one time-varying stimulation parameter of the discrete dynamic pattern by modulating the selected parameter using the discretized modulation function.

In Example 2, the subject matter of Example 1 may optionally be configured to include a programming device configured to program the stimulation device. The programming device includes the programming control circuit and the stimulation programming circuit.

In Example 3, the subject matter of Example 2 may optionally be configured to further include the stimulation device. The stimulation device includes an implantable neurostimulator. The programming device includes an external programmer configured to communicate with the implantable neurostimulator wirelessly.

In Example 4, the subject matter of any one or any combination of Examples 1 to 3 may optionally be configured such that the modulation function generator is configured to receive one or more discretization parameters of the discretization information and to determine the discretized modulation function according to the one or more discretization parameters.

In Example 5, the subject matter of Example 4 may optionally be configured such that the modulation function generator includes discretization circuitry configured to receive a continuous function, to receive the one or more discretization parameters, and to generate the discretized modulation function by discretizing the continuous function using the one or more discretization parameters.

In Example 6, the subject matter of any one or any combination of Examples 4 and 5 may optionally be configured such that the modulation function generator is configured to receive a step number of the one or more discretization parameters and to determine the discretized modulation function using the received step number. The step number defines a number of steps in transitioning between a lower amplitude and an upper amplitude specified for the discretized modulation function.

In Example 7, the subject matter of any one or any combination of Examples 4 to 6 may optionally be configured such that the modulation function generator is configured to receive a step size of the one or more discretization parameters and to determine the discretized modulation function using the received step size. The step size defines a magnitude of each step of the steps in transitioning between the lower amplitude and the upper amplitude specified for the discretized modulation function.

In Example 8, the subject matter of any one or any combination of Examples 4 to 7 may optionally be configured such that the modulation function generator is configured to receive a discretization parameter of the one or more discretization parameters that is a function of the patient's perception threshold and to determine the discretized modulation function using the received discretization parameter. The perception threshold corresponds to a minimum value of the selected parameter for the patient to perceive the neurostimulation being delivered.

In Example 9, the subject matter of any one or any combination of Examples 4 to 7 may optionally be configured such that the modulation function generator is configured to receive a discretization parameter of the one or more discretization parameters that has multiple values corresponding to ranges of the selected parameter and to determine the discretized modulation function using the received discretization parameter.

In Example 10, the subject matter of any one or any combination of Examples 4 to 7 may optionally be configured such that the modulation function generator is configured to determine a discretization parameter of the one or more discretization parameters based on settings of one or more stimulation parameters of the stimulation parameters other than the selected parameter and to determine the discretized modulation function using the determined discretization parameter.

In Example 11, the subject matter of any one or any combination of Examples 4 to 7 may optionally be configured such that the modulation function generator is configured to determine a discretization parameter of the one or more discretization parameters based on a signal sensed from the patient and to determine the discretized modulation function using the determined discretization parameter.

In Example 12, the subject matter of any one or any combination of Examples 4 to 7 may optionally be configured such that the modulation function generator is configured to determine a discretization parameter of the one or more discretization parameters based on an amount of charge delivered to the patient by the delivery of the neurostimulation and to determine the discretized modulation function using the determined discretization parameter.

In Example 13, the subject matter of any one or any combination of Examples 1 to 12 may optionally be configured such that the stimulation programming circuit is configured to determine a neurostimulation program including multiple dynamic stimulation patterns and a schedule specifying a time period for each dynamic stimulation pattern of the multiple dynamic stimulation patterns to be applied for controlling the delivery of the neurostimulation.

In Example 14, the subject matter of Example 13 may optionally be configured such that the dynamic pattern composer is further configured to determine a continuous dynamic pattern of the multiple dynamic stimulation patterns, the modulation function generator is further configured to generate a continuous modulation function that is a continuously time-varying function, and the parameter modulator is configured to select a parameter from the stimulation parameters of the continuous dynamic pattern and to produce the at least one time-varying stimulation parameter of the continuous dynamic pattern by modulating the selected parameter using the continuous modulation function.

In Example 15, the subject matter of Example 14 may optionally be configured such that the multiple dynamic stimulation patterns include the discrete dynamic pattern and the continuous dynamic pattern, and the schedule specifies time periods for the discrete dynamic pattern and the continuous dynamic pattern that toggle between the discrete dynamic pattern and the continuous dynamic pattern.

In Example 16, a method for delivering neurostimulation from a stimulation device to a patient is provided. The method may include determining one or more dynamic stimulation patterns each defined by stimulation parameters including at least one time-varying stimulation parameter. The one or more dynamic stimulation patterns may include a discrete dynamic pattern. The determination of the discrete dynamic pattern may include receiving discretization information, determining a discretized modulation function using the received discretization information, selecting a parameter from the stimulation parameters of the discrete dynamic pattern, and producing the at least one time-varying stimulation parameter of the discrete dynamic pattern by modulating the selected parameter using the discretized modulation function. The discretized modulation function is a discretely time-varying function. The method may further include determining a neurostimulation program including the one or more dynamic stimulation patterns and generating information for programming the stimulation device to control the delivery of the neurostimulation according to the neurostimulation program.

In Example 17, the subject matter of receiving the discretization information as found Example 16 may optionally include receiving one or more discretization parameters including at least one of a step number or a step size, The step number defines a number of steps in transitioning between a lower amplitude and an upper amplitude specified for the discretized modulation function. The step size defines a magnitude of each step of the steps.

In Example 18, the subject matter of receiving the one or more discretization parameters as found Example 17 may optionally include receiving a discretization parameter being a function of the patient's perception threshold, the perception threshold corresponding to a maximum value of the selected parameter tolerable by the patient.

In Example 19, the subject matter of receiving the one or more discretization parameters as found Example 17 may optionally include receiving a discretization parameter having multiple values corresponding to ranges of the selected parameter.

In Example 20, the subject matter of the determination of the discrete dynamic pattern as found Example 17 may optionally further include determining a discretization parameter of the one or more discretization parameters based on settings of one or more stimulation parameters of the stimulation parameters other than the selected parameter.

In Example 21, the subject matter of the determination of the discrete dynamic pattern as found Example 17 may optionally further include determining a discretization parameter of the one or more discretization parameters based on a signal sensed from the patient.

In Example 22, the subject matter of the determination of the discrete dynamic pattern as found Example 17 may optionally further include determining a discretization parameter of the one or more discretization parameters based on an amount of charge delivered to the patient by the delivery of the neurostimulation.

In Example 23, the subject matter of determining the neurostimulation program as found in any one or any combination of Examples 16 to 22 may optionally include determining a neurostimulation program including multiple dynamic stimulation patterns including the discrete dynamic pattern and a continuous dynamic pattern, and the subject matter of determining the one or more dynamic stimulation patterns as found in any one or any combination of Examples 16 to 22 may optionally include determining the continuous dynamic pattern, including generating a continuous modulation function that is a continuously time-varying function, selecting a parameter from the stimulation parameters of the continuous dynamic pattern, and producing the at least one time-varying stimulation parameter of the continuous dynamic pattern by modulating the selected parameter using the continuous modulation function.

In Example 24, the subject matter of determining the neurostimulation program as found in Example 23 may optionally include scheduling time periods for the discrete dynamic pattern and the continuous dynamic pattern such that the discrete dynamic pattern and the continuous dynamic pattern are applied at different times.

In Example 25, a non-transitory computer-readable storage medium including instructions is provided. The instructions, which when executed by a system, may cause the system to perform a method for delivering neurostimulation from a stimulation device to a patient The method may include determining one or more dynamic stimulation patterns each defined by stimulation parameters including at least one time-varying stimulation parameter. The one or more dynamic stimulation patterns includes a discrete dynamic pattern. The determination of the discrete dynamic pattern including receiving discretization information, determining a discretized modulation function using the received discretization information, selecting a parameter from the stimulation parameters of the discrete dynamic pattern, and producing the at least one time-varying stimulation parameter of the discrete dynamic pattern by modulating the selected parameter using the discretized modulation function. The discretized modulation function is a discretely time-varying function. The method may further include determining a neurostimulation program including the one or more dynamic stimulation patterns and generating information for programming the stimulation device to control the delivery of the neurostimulation according to the neurostimulation program.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 illustrates an embodiment of a neurostimulation system.

FIG. 2 illustrates an embodiment of a stimulation device and a lead system, such as may be implemented in the neurostimulation system of FIG. 1.

FIG. 3 illustrates an embodiment of a programming device, such as may be implemented in the neurostimulation system of FIG. 1.

FIG. 4 illustrates an embodiment of an implantable pulse generator (IPG) and an implantable lead system, such as an example implementation of the stimulation device and lead system of FIG. 2.

FIG. 5 illustrates an embodiment of an IPG and an implantable lead system, such as the IPG and lead system of FIG. 4, arranged to provide neurostimulation to a patient.

FIG. 6 illustrates an embodiment of portions of a neurostimulation system.

FIG. 7 illustrates an embodiment of an implantable stimulator and one or more leads of an implantable neurostimulation system, such as the implantable neurostimulation system of FIG. 6.

FIG. 8 illustrates an embodiment of an external programming device of an implantable neurostimulation system, such as the implantable neurostimulation system of FIG. 6.

FIG. 9 illustrates an embodiment of a system for programming neurostimulation with one or more dynamic stimulation patterns including a discrete dynamic pattern.

FIGS. 10A-C illustrate examples of stimulation patterns, with FIG. 10A illustrating a tonic stimulation pattern, FIG. 10B illustrating a continuous dynamic pattern, and FIG. 10C illustrating a discrete dynamic pattern.

FIGS. 11A-C illustrate examples of waveforms in an embodiment of producing a discrete dynamic pattern, with FIG. 11A illustrating a continuous function, FIG. 11B illustrating a discretized modulation function resulting from discretization of the continuous function, and FIG. 11C illustrating the discrete stimulation pattern resulting from modulating a pulse amplitude using the discretized modulation function.

FIGS. 12A-B illustrate examples of discrete dynamic patterns resulting from modulating a pulse amplitude using discretized modulation functions having different step numbers, with FIG. 12A illustrating an example with step number 4 and FIG. 12B illustrating an example with step number 6.

FIG. 13 illustrates an embodiment of a method for programming a stimulation device for delivering neurostimulation to a patient according to one or more dynamic stimulation patterns including a discrete dynamic pattern.

FIG. 14 illustrates an embodiment of a method for determining the discrete dynamic pattern in the method of FIG. 13.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized, and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses, among other things, a neurostimulation system that can deliver neurostimulation to a patient and control the delivery of the neurostimulation according to one or more dynamic stimulation patterns that can each be a discrete dynamic pattern. Dynamic stimulation patterns can more closely resemble natural neural signals than tonic stimulation patterns. One problem associated with tonic stimulation patterns is habitation, which is a decrease in a patient's response to neurostimulation as a result of prolonged neurostimulation with time-invariant stimulation parameters. Habitation does not occur with the patient's natural neural activities. Thus, dynamic stimulation patterns can be applied for preventing progressive loss of efficacy of a neurostimulation therapy due to habituation.

In this document, a “stimulation pattern” includes a sequence (or “train”) of neurostimulation pulses defined by stimulation parameters. A “tonic stimulation pattern” (also known as “tonic pulse sequence”, “tonic pulse train”, and the like) includes a pattern of neurostimulation pulses defined by stimulation parameters having constant values. A “dynamic stimulation pattern” (also known as “dynamic pulse sequence”, “dynamic pulse train”, and the like) includes a pattern of neurostimulation pulses defined by stimulation parameters including at least one stimulation parameter having a time-varying value. It is noted that in a tonic stimulation pattern, all the stimulation parameters have constant (i.e., not time-varying) values, while in a dynamic stimulation pattern, the stimulation parameters can each have a constant value or a time-varying value. In other words, at least one of the stimulation parameters of a dynamic stimulation pattern has a time-varying value, while the other stimulation parameters can each have a constant or time-varying value. One example of a dynamic stimulation pattern includes a “modulated stimulation pattern” (also known as “modulated pulse sequence”, “modulated pulse train, and the like), in which one or more of the stimulation parameters are each modulated by a modulation function. Examples of such modulated pulse sequence are discussed in U.S. patent application Ser. No. 17/530,236, “METHOD AND APPARATUS FOR GENERATING MODULATED NEUROSTIMULATION PULSE SEQUENCE”, filed on Nov. 18, 2021, published as US 2022/0184400 A1, assigned to Boston Scientific Neuromodulation Corporation, which is incorporated herein by reference in its entirety.

Neurostimulation with dynamic stimulation patterns defined by one or more continuously time-varying stimulation parameters (e.g., pulse amplitude, pulse width, and/or pulse frequency) has been applied to treat patients. Delivery of neurostimulation pulses controlled using stimulation parameters programmed to constant (not time-varying) values has been effective in treating various conditions, such as in treating pain using SCS. Dynamic stimulation patterns defined by time-varying stimulation parameters can be applied to produce more biomimetic effects on the nervous system and/or to improve therapeutic effects of SCS. An example of such dynamic stimulation patterns includes a modulated stimulation pattern in which a pulse amplitude is modulated by a continuous sinusoidal function as the modulation function. The sinusoidal function can provide a slow and gradual varying stimulation intensity that results in a pleasant or tolerable sensation for the patients. However, the frequent (e.g., pulse by pulse) change of stimulation parameter values consumes a significant amount of power and computational and storage resources, which are all limited particularly when the neurostimulation is delivered using implantable devices. Also, the pleasant or tolerable sensation may not be a practical benefit when the neurostimulation is intended to be a sub-perceptive therapy (i.e., the patients do not perceive the stimulation).

The present subject matter allows for control of delivery of neurostimulation using discrete dynamic patterns. Dynamic stimulation patterns can include continuous dynamic patterns and discrete dynamic patterns. A “continuous dynamic pattern” is a dynamic stimulation pattern defined by stimulation parameters including at least one stimulation parameter having a continuously time-varying value. A “discrete dynamic pattern” is a dynamic stimulation pattern defined by stimulation parameters including at least one stimulation parameter having a discretized time-varying value. For example, the discretized time-varying value can result from modulating the stimulation parameter (e.g., pulse amplitude, pulse width, or pulse frequency) using a discretized modulation function (e.g., a discretized sinusoidal, triangular, positive ramp, or negative ramp function). In various embodiments, a discretized modulation function can be a quantized version of the corresponding continuous modulation function. In other words, the discretized modulation function can have one or more time varying parameters that vary in discrete steps. The discretized modulation function (e.g., a discretized sinusoidal function) approximates its continuous modulation function counterpart (e.g., a continuous sinusoidal function). The discrete dynamic pattern generated using the discretized modulation function approximates its continuous dynamic pattern counterpart generated using the continuous modulation function counterpart. Such approximation may not significantly affect the efficacy of the neurostimulation and may not affect the patient's perception of the neurostimulation, particularly when the neurostimulation is programmed to be a sub-perceptive therapy.

Neurostimulation with dynamic stimulation patterns generally consumes more power than their tonic stimulation pattern counterparts due to the additional processing power required for frequent parameter value changes. The use of discrete dynamic patterns can reduce the additional processing power by reducing the frequency of parameter value changes, when compared to their continuous dynamic pattern counterparts. Neurostimulation with dynamic stimulation patterns generally requires more memory than their tonic stimulation pattern counterparts due to the changes of values of each time-varying parameter. Discrete dynamic patterns can reduce the amount of value changes when compared to their continuous dynamic pattern counterparts. Discrete dynamic patterns should not significantly affect therapy efficacy and patients' perception (e.g., intensity of paresthesia), especially when sub-perceptive therapies are applied, when compared to their continuous dynamic pattern counterparts.

The delivery of the neurostimulation according to the present subject matter includes delivering neurostimulation pulses. In various embodiments, the present neuromodulation system can include an implantable device configured to deliver neurostimulation (also referred to as neuromodulation) therapies, such as spinal cord stimulation (SCS), deep brain stimulation (DBS), peripheral nerve stimulation (PNS), and vagus nerve stimulation (VNS), and one or more external devices configured to program or adjust the implantable device for its operations and monitor the performance of the implantable device. In this document, unless noted otherwise, a “patient” includes a person receiving treatment delivered from, and/or monitored using, a neurostimulation system according to the present subject matter. A “user” includes a physician, other caregiver who examines and/or treats the patient using the neurostimulation system, or other person who participates in the examination and/or treatment of the patient using the neurostimulation system (e.g., a technically trained representative, a field clinical engineer, a clinical researcher, or a field specialist from the manufacturer of the neurostimulation system).

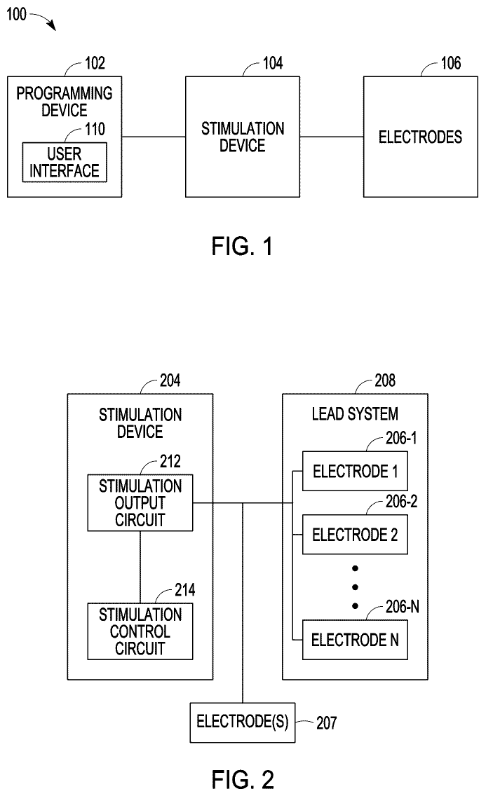

FIG. 1 illustrates an embodiment of a neurostimulation system 100. System 100 includes electrodes (also referred to as contacts) 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using a plurality of stimulation parameters, such as stimulation parameters specifying a pattern of the electrical pulses and a selection of electrodes through which each of the electrical pulses is delivered. In various embodiments, at least some parameters of the plurality of stimulation parameters are programmable by a user, such as a physician or other caregiver who treats the patient using system 100. Programming device 102 provides the user with accessibility to the user-programmable parameters. In various embodiments, programming device 102 is configured to be communicatively coupled to stimulation device via a wired or wireless link.

In various embodiments, programming device 102 can include a user interface 110 that allows the user to control the operation of system 100 and monitor the performance of system 100 as well as conditions of the patient including responses to the delivery of the neurostimulation. The user can control the operation of system 100 by setting and/or adjusting values of the user-programmable parameters.

In various embodiments, user interface 110 can include a graphical user interface (GUI) that allows the user to set and/or adjust the values of the user-programmable parameters by creating and/or editing graphical representations of various waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In various embodiments, neurostimulation pulses for a stimulation period (such as the duration of a therapy session) may be delivered to multiple stimulation fields.

In various embodiments, system 100 can be configured for neurostimulation applications. User interface 110 can be configured to allow the user to control the operation of system 100 for neurostimulation. For example, system 100 as well as user interface 110 can be configured for spinal cord stimulation (SCS) applications. Such SCS configuration includes various features that may simplify the task of the user in programming stimulation device 104 for delivering SCS to the patient, such as the features discussed in this document.

FIG. 2 illustrates an embodiment of a stimulation device 204 and a lead system 208, such as may be implemented in neurostimulation system 100. Stimulation device 204 represents an example of stimulation device 104 and includes a stimulation output circuit 212 and a stimulation control circuit 214. Stimulation output circuit 212 produces and delivers neurostimulation pulses. Stimulation control circuit 214 controls the delivery of the neurostimulation pulses from stimulation output circuit 212 using the plurality of stimulation parameters, which specifies a pattern of the neurostimulation pulses. Lead system 208 includes one or more leads each configured to be electrically connected to stimulation device 204 and a plurality of electrodes 206 (also referred to as an electrode array in this document) distributed in the one or more leads. The plurality of electrodes 206 includes electrode 206-1, electrode 206-2, . . . electrode 206-N, each being a single electrically conductive contact providing for an electrical interface between stimulation output circuit 212 and tissue of the patient (and therefore also referred to as a contact), where N≥2. The neurostimulation pulses are each delivered from stimulation output circuit 212 through a set of electrodes selected from electrodes 206. In various embodiments, the neurostimulation pulses may include one or more individually defined pulses, and the set of electrodes may be individually definable by the user for each of the individually defined pulses or each of collections of pulse intended to be delivered using the same combination of electrodes. In various embodiments, one or more additional electrodes 207 (each of which may be referred to as a reference electrode) can be electrically connected to stimulation device 204, such as one or more electrodes each being a portion of or otherwise incorporated onto a housing of stimulation device 204. Monopolar stimulation uses a monopolar electrode configuration with one or more electrodes selected from electrodes 206 and at least one electrode from electrode(s) 207. Bipolar stimulation uses a bipolar electrode configuration with two electrodes selected from electrodes 206 and none electrode(s) 207. Multipolar stimulation uses a multipolar electrode configuration with multiple (two or more) electrodes selected from electrodes 206 and none of electrode(s) 207.

In various embodiments, the number of leads and the number of electrodes on each lead depend on, for example, the distribution of target(s) of the neurostimulation and the need for controlling the distribution of electric field at each target. In one embodiment, lead system 208 includes 2 leads each having 8 electrodes.

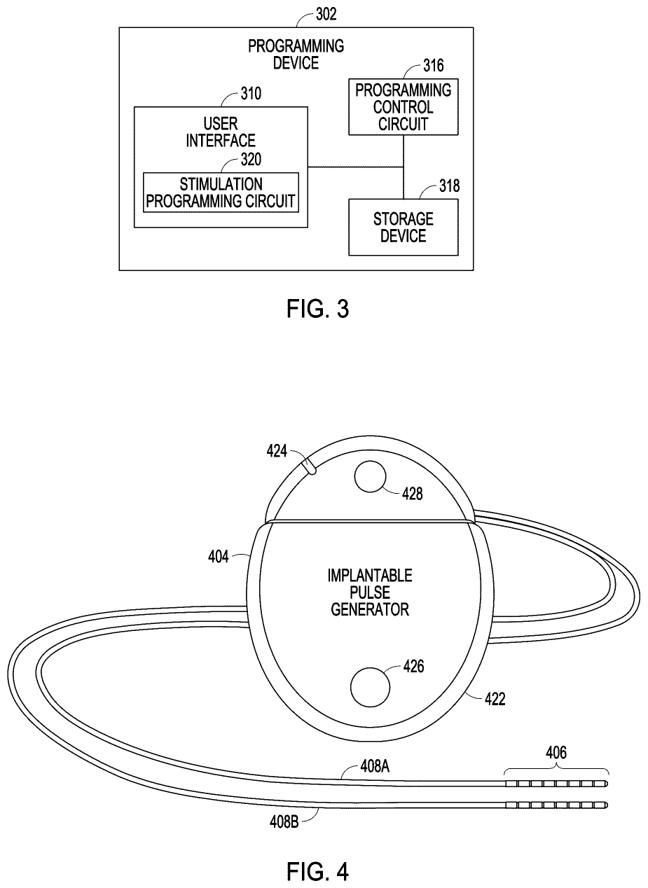

FIG. 3 illustrates an embodiment of a programming device 302, such as may be implemented in neurostimulation system 100. Programming device 302 represents an example of programming device 102 and includes a storage device 318, a programming control circuit 316, and a user interface 310. Programming control circuit 316 generates the plurality of stimulation parameters that controls the delivery of the neurostimulation pulses according to a specified neurostimulation program that can define, for example, stimulation waveform and electrode configuration. User interface 310 represents an example of user interface 110 and includes a stimulation programming circuit 320. Storage device 318 stores information used by programming control circuit 316 and stimulation programming circuit 320, such as information about a stimulation device that relates the neurostimulation program to the plurality of stimulation parameters. In various embodiments, stimulation programming circuit 320 can be configured to support one or more functions allowing for programming of stimulation devices, such as stimulation device 104 including its various embodiments as discussed in this document, to control delivery of neurostimulation according to the present subject matter (e.g., delivering neurostimulation pulses according to one or more dynamic stimulation patterns including at least one discrete stimulation pattern).

In various embodiments, user interface 310 can allow for definition of a pattern of neurostimulation pulses for delivery during a neurostimulation therapy session by creating and/or adjusting one or more stimulation waveforms using a graphical method. The definition can also include definition of one or more stimulation fields each associated with one or more pulses in the pattern of neurostimulation pulses. As used in this document, a “neurostimulation program” or “stimulation program” can include the pattern of neurostimulation pulses defined using the one or more stimulation waveforms and the one or more stimulation fields, or at least various spatial, temporal, and/or informational aspects or parameters of the pattern of neurostimulation pulses including the one or more stimulation waveforms and the one or more stimulation fields. In various embodiments, user interface 310 includes a GUI that allows the user to define the pattern of neurostimulation pulses and perform other functions using graphical methods. In this document, “neurostimulation programming” can include the definition of the one or more stimulation waveforms, including the definition of one or more stimulation fields.

In various embodiments, circuits of neurostimulation system 100, including its various embodiments discussed in this document, may be implemented using a combination of hardware and software. For example, the circuit of user interface 110, stimulation control circuit 214, programming control circuit 316, and stimulation programming circuit 320, including their various embodiments discussed in this document, can be implemented using an application-specific circuit constructed to perform one or more particular functions and/or a general-purpose circuit programmed to perform such function(s). Such a general-purpose circuit includes, but is not limited to, a microprocessor or a portion thereof, a microcontroller or portions thereof, and a programmable logic circuit or a portion thereof.

FIG. 4 illustrates an embodiment of an implantable pulse generator (IPG) 404 and an implantable lead system 408. IPG 404 represents an example implementation of stimulation device 204. Lead system 408 represents an example implementation of lead system 208. As illustrated in FIG. 4, IPG 404 that can be coupled to implantable leads 408A and 408B at a proximal end of each lead. The distal end of each lead includes electrodes 406 for contacting a tissue site targeted for electrical neurostimulation. As illustrated in FIG. 4, leads 408A and 408B each include 8 electrodes 406 at the distal end. The number and arrangement of leads 408A and 408B and electrodes 406 as shown in FIG. 4 are only an example, and other numbers and arrangements are possible. In various embodiments, the electrodes are ring electrodes. In various embodiments applying DBS or SCS, the implantable leads and electrodes may be configured by shape and size to provide electrical neurostimulation energy to a neuronal target included in the patient's brain or configured to provide electrical neurostimulation energy to target nerve cells in the patient's spinal cord.

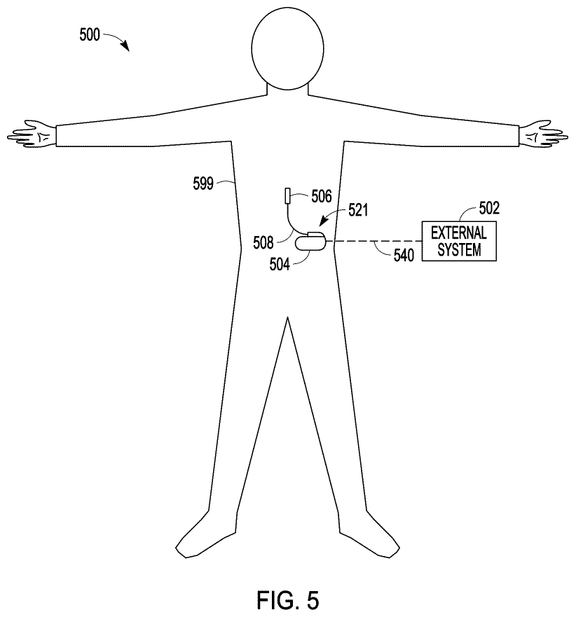

FIG. 5 illustrates an implantable neurostimulation system 500 and portions of an environment in which system 500 may be used. System 500 includes an implantable system 521, an external system 502, and a telemetry link 540 providing for wireless communication between implantable system 521 and external system 502. Implantable system 521 is illustrated in FIG. 5 as being implanted in the patient's body 599.

Implantable system 521 includes an implantable stimulator (also referred to as an implantable pulse generator, or IPG) 504, a lead system 508, and electrodes 506, which represent an example of stimulation device 204, lead system 208, and electrodes 206, respectively. External system 502 represents an example of programming device 302. In various embodiments, external system 502 includes one or more external (non-implantable) devices each allowing the user and/or the patient to communicate with implantable system 521. In some embodiments, external 502 includes a programming device intended for the user to initialize and adjust settings for implantable stimulator 504 and a remote control device intended for use by the patient. For example, the remote control device may allow the patient to turn implantable stimulator 504 on and off and/or adjust certain patient-programmable parameters of the plurality of stimulation parameters.

The sizes and shapes of the elements of implantable system 521 and their location in body 599 are illustrated by way of example and not by way of restriction. An implantable system is discussed as a specific application of the programming according to various embodiments of the present subject matter. In various embodiments, the present subject matter may be applied in programming any type of stimulation device that uses electrical pulses as stimuli, regarding less of stimulation targets in the patient's body and whether the stimulation device is implantable.

Returning to FIG. 4, the IPG 404 can include a hermetically-sealed IPG case 422 to house the electronic circuitry of IPG 404. IPG 404 can include an electrode 426 formed on IPG case 422. IPG 404 can include an IPG header 424 for coupling the proximal ends of leads 408A and 408B. IPG header 424 may optionally also include an electrode 428. Electrodes 426 and/or 428 represent embodiments of electrode(s) 207 and may each be referred to as a reference electrode. Neurostimulation energy can be delivered in a monopolar (also referred to as unipolar) mode using electrode 426 or electrode 428 and one or more electrodes selected from electrodes 406. Neurostimulation energy can be delivered in a bipolar mode using a pair of electrodes of the same lead (lead 408A or lead 408B). Neurostimulation energy can be delivered in an extended bipolar mode using one or more electrodes of a lead (e.g., one or more electrodes of lead 408A) and one or more electrodes of a different lead (e.g., one or more electrodes of lead 408B).

The electronic circuitry of IPG 404 can include a control circuit that controls delivery of the neurostimulation energy. The control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

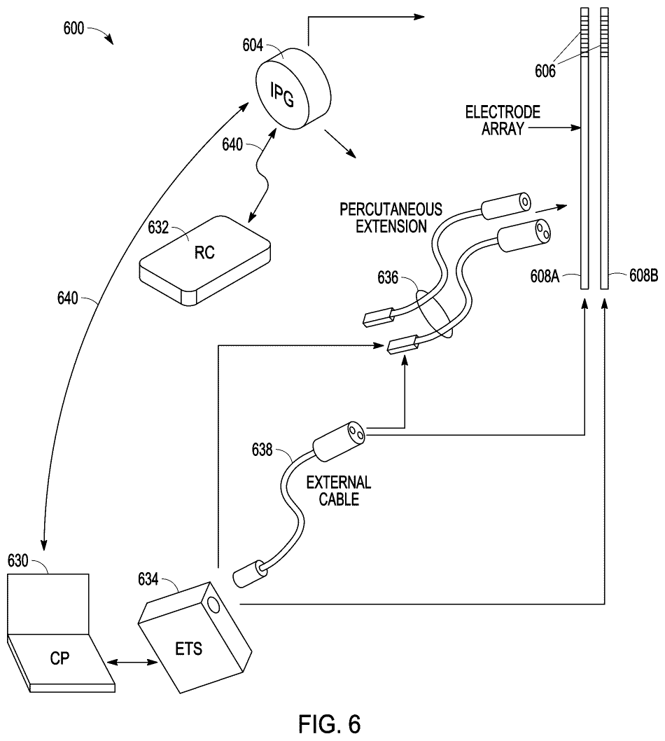

FIG. 6 illustrates an embodiment of portions of a neurostimulation system 600. System 600 includes an IPG 604, implantable neurostimulation leads 608A and 608B, an external remote controller (RC) 632, a clinician's programmer (CP) 630, and an external trial stimulator (ETS, also referred to as external trial modulator, ETM) 634. IPG 604 may be electrically coupled to leads 608A and 608B directly or through percutaneous extension leads 636. ETS 634 may be electrically connectable to leads 608A and 608B via one or both of percutaneous extension leads 636 and/or external cable 638. System 600 represents an example of system 100, with IPG 604 representing an embodiment of stimulation device 104, electrodes 606 of leads 608A and 608B representing electrodes 106, and CP 630, RC 632, and ETS 634 collectively representing programming device 102.

ETS 634 may be standalone or incorporated into CP 630. ETS 634 may have similar pulse generation circuitry as IPG 604 to deliver neurostimulation energy according to specified modulation parameters as discussed above. ETS 634 is an external device that is typically used as a preliminary stimulator after leads 408A and 408B have been implanted and used prior to stimulation with IPG 604 to test the patient's responsiveness to the stimulation that is to be provided by IPG 604. Because ETS 634 is external it may be more easily configurable than IPG 604.

CP 630 can configure the neurostimulation provided by ETS 634. If ETS 634 is not integrated into CP 630, CP 630 may communicate with ETS 634 using a wired connection (e.g., over a USB link) or by wireless telemetry using a wireless communications link 640. CP 630 also communicates with IPG 604 using a wireless communications link 640.

An example of wireless telemetry is based on inductive coupling between two closely-placed coils using the mutual inductance between these coils. This type of telemetry is referred to as inductive telemetry or near-field telemetry because the coils must typically be closely situated for obtaining inductively coupled communication. IPG 604 can include the first coil and a communication circuit. CP 630 can include or otherwise electrically connected to the second coil such as in the form of a wand that can be place near IPG 604. Another example of wireless telemetry includes a far-field telemetry link, also referred to as a radio frequency (RF) telemetry link. A far-field, also referred to as the Fraunhofer zone, refers to the zone in which a component of an electromagnetic field produced by the transmitting electromagnetic radiation source decays substantially proportionally to 1/r, where r is the distance between an observation point and the radiation source. Accordingly, far-field refers to the zone outside the boundary of r=λ/2π, where λ is the wavelength of the transmitted electromagnetic energy. In one example, a communication range of an RF telemetry link is at least six feet but can be as long as allowed by the particular communication technology. RF antennas can be included, for example, in the header of IPG 604 and in the housing of CP 630, eliminating the need for a wand or other means of inductive coupling. An example is such an RF telemetry link is a Bluetooth® wireless link.

CP 630 can be used to set modulation parameters for the neurostimulation after IPG 604 has been implanted. This allows the neurostimulation to be tuned if the requirements for the neurostimulation change after implantation. CP 630 can also upload information from IPG 604.

RC 632 also communicates with IPG 604 using a wireless link 640. RC 632 may be a communication device used by the user or given to the patient. RC 632 may have reduced programming capability compared to CP 630. This allows the user or patient to alter the neurostimulation therapy but does not allow the patient full control over the therapy. For example, the patient may be able to increase the amplitude of neurostimulation pulses or change the time that a preprogrammed stimulation pulse train is applied. RC 632 may be programmed by CP 630. CP 630 may communicate with the RC 632 using a wired or wireless communications link. In some embodiments, CP 630 can program RC 632 when remotely located from RC 632. In various embodiments, RC 632 can be a dedicated device or a general-purpose device configured to perform the functions of RC 632, such as a smartphone, a tablet computer, or other smart/mobile device.

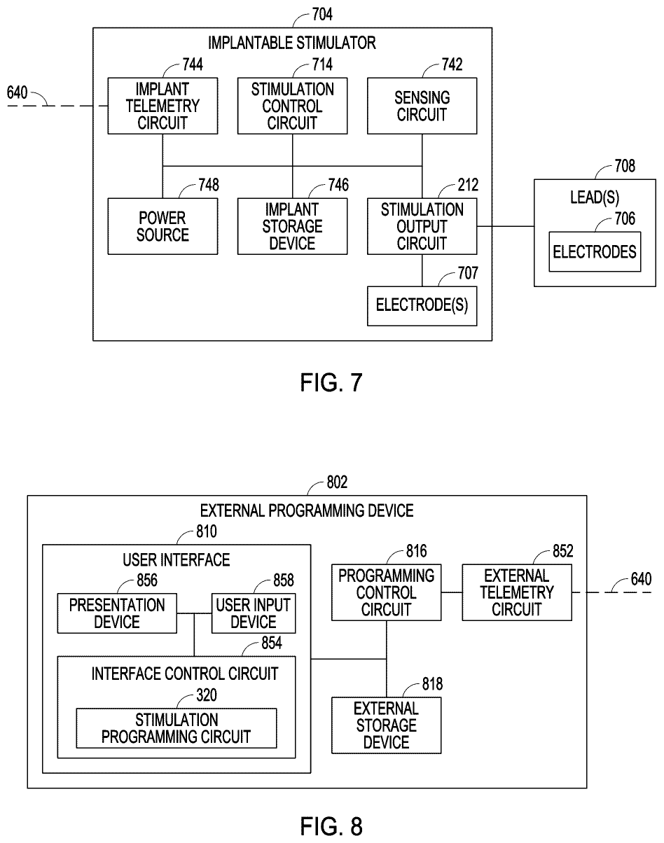

FIG. 7 illustrates an embodiment of implantable stimulator 704 and one or more leads 708 of an implantable neurostimulation system, such as implantable system 600. Implantable stimulator 704 represents an example of stimulation device 104 or 204 and may be implemented, for example, as IPG 604. Lead(s) 708 represents an example of lead system 208 and may be implemented, for example, as implantable leads 608A and 608B. Lead(s) 708 includes electrodes 706, which represents an example of electrodes 106 or 206 and may be implemented as electrodes 606.

Implantable stimulator 704 may include a sensing circuit 742 that provides the stimulator with a sensing capability, stimulation output circuit 212, a stimulation control circuit 714, an implant storage device 746, an implant telemetry circuit 744, a power source 748, and one or more electrodes 707. Sensing circuit 742 can one or more physiological signals for purposes of patient monitoring and/or feedback control of the neurostimulation. In various embodiments, sensing circuit 742 can sense one or more electrospinogram (ESG) signals using electrodes placed over or under the dura of the spinal cord, such as electrodes 706 (which can include epidural and/or intradural electrodes). In addition to one or more ESG signals, examples of the one or more physiological signals include neural and other signals each indicative of a condition of the patient that is treated by the neurostimulation and/or a response of the patient to the delivery of the neurostimulation. Stimulation output circuit 212 is electrically connected to electrodes 706 through one or more leads 708 as well as electrodes 707 and delivers each of the neurostimulation pulses through a set of electrodes selected from electrodes 706 and electrode(s) 707. Stimulation control circuit 714 represents an example of stimulation control circuit 214 and controls the delivery of the neurostimulation pulses using the plurality of stimulation parameters specifying the pattern of neurostimulation pulses. In one embodiment, stimulation control circuit 714 controls the delivery of the neurostimulation pulses using the one or more sensed physiological signals. Implant telemetry circuit 744 provides implantable stimulator 704 with wireless communication with another device such as CP 630 and RC 632, including receiving values of the plurality of stimulation parameters from the other device. Implant storage device 746 can store one or more neurostimulation programs and values of the plurality of stimulation parameters for each of the one or more neurostimulation programs. Power source 748 provides implantable stimulator 704 with energy for its operation. In one embodiment, power source 748 includes a battery. In one embodiment, power source 748 includes a rechargeable battery and a battery charging circuit for charging the rechargeable battery. Implant telemetry circuit 744 may also function as a power receiver that receives power transmitted from an external device through an inductive couple. Electrode(s) 707 allow for delivery of the neurostimulation pulses in the monopolar mode. Examples of electrode(s) 707 include electrode 426 and electrode 418 in IPG 404 as illustrated in FIG. 4.

In one embodiment, implantable stimulator 704 is used as a master database. A patient implanted with implantable stimulator 704 (such as may be implemented as IPG 604) may therefore carry patient information needed for his or her medical care when such information is otherwise unavailable. Implant storage device 746 is configured to store such patient information. For example, the patient may be given a new RC 632 (e.g., by installing a new application in a smart device such as a smartphone) and/or travel to a new clinic where a new CP 630 is used to communicate with the device implanted in him or her. The new RC 632 and/or CP 630 can communicate with implantable stimulator 704 to retrieve the patient information stored in implant storage device 746 through implant telemetry circuit 744 and wireless communication link 640 and allow for any necessary adjustment of the operation of implantable stimulator 704 based on the retrieved patient information. In various embodiments, the patient information to be stored in implant storage device 746 may include, for example, positions of lead(s) 708 and electrodes 706 relative to the patient's anatomy (transformation for fusing computerized tomogram (CT) of post-operative lead placement to magnetic resonance imaging (MRI) of the brain), clinical effect map data, objective measurements using quantitative assessments of symptoms (for example using micro-electrode recording, accelerometers, and/or other sensors), and/or any other information considered important or useful for providing adequate care for the patient. In various embodiments, the patient information to be stored in implant storage device 746 may include data transmitted to implantable stimulator 704 for storage as part of the patient information and data acquired by implantable stimulator 704, such as by using sensing circuit 742.

In various embodiments, sensing circuit 742 (if included), stimulation output circuit 212, stimulation control circuit 714, implant telemetry circuit 744, implant storage device 746, and power source 748 are encapsulated in a hermetically sealed implantable housing or case, and electrode(s) 707 are formed or otherwise incorporated onto the case. In various embodiments, lead(s) 708 are implanted such that electrodes 706 are placed on and/or around one or more targets to which the neurostimulation pulses are to be delivered, while implantable stimulator 704 is subcutaneously implanted and connected to lead(s) 708 at the time of implantation.

FIG. 8 illustrates an embodiment of an external programming device 802 of an implantable neurostimulation system, such as system 600. External programming device 802 represents an example of programming device 102 or 302, and may be implemented, for example, as CP 630 and/or RC 632. External programming device 802 includes an external telemetry circuit 852, an external storage device 818, a programming control circuit 816, and a user interface 810.

External telemetry circuit 852 provides external programming device 802 with wireless communication with another device such as implantable stimulator 704 via wireless communication link 640, including transmitting the plurality of stimulation parameters to implantable stimulator 704 and receiving information including the patient data from implantable stimulator 704. In one embodiment, external telemetry circuit 852 also transmits power to implantable stimulator 704 through an inductive couple.

In various embodiments, wireless communication link 640 can include an inductive telemetry link (near-field telemetry link) and/or a far-field telemetry link (RF telemetry link). This can allow for patient mobility during programming and assessment when needed. For example, wireless communication link 640 can include at least a far-field telemetry link that allows for communications between external programming device 802 and implantable stimulator 704 over a relative long distance, such as up to about 20 meters. External telemetry circuit 852 and implant telemetry circuit 744 each include an antenna and RF circuitry configured to support such wireless telemetry.

External storage device 818 stores one or more stimulation waveforms for delivery during a neurostimulation therapy session, such as a DBS or SCS therapy session, as well as various parameters and building blocks for defining one or more waveforms. The one or more stimulation waveforms may each be associated with one or more stimulation fields and represent a pattern of neurostimulation pulses to be delivered to the one or more stimulation field during the neurostimulation therapy session. In various embodiments, each of the one or more stimulation waveforms can be selected for modification by the user and/or for use in programming a stimulation device such as implantable stimulator 704 to deliver a therapy. In various embodiments, each waveform in the one or more stimulation waveforms is definable on a pulse-by-pulse basis, and external storage device 818 may include a pulse library that stores one or more individually definable pulse waveforms each defining a pulse type of one or more pulse types. External storage device 818 also stores one or more individually definable stimulation fields. Each waveform in the one or more stimulation waveforms is associated with at least one field of the one or more individually definable stimulation fields. Each field of the one or more individually definable stimulation fields is defined by a set of electrodes through which a neurostimulation pulse is delivered. In various embodiments, each field of the one or more individually definable fields is defined by the set of electrodes through which the neurostimulation pulse is delivered and a current distribution of the neurostimulation pulse over the set of electrodes. In one embodiment, the current distribution is defined by assigning a fraction of an overall pulse amplitude to each electrode of the set of electrodes. Such definition of the current distribution may be referred to as “fractionalization” in this document. In another embodiment, the current distribution is defined by assigning an amplitude value to each electrode of the set of electrodes. For example, the set of electrodes may include 2 electrodes used as the anode and an electrode as the cathode for delivering a neurostimulation pulse having a pulse amplitude of 4 mA. The current distribution over the 2 electrodes used as the anode needs to be defined. In one embodiment, a percentage of the pulse amplitude is assigned to each of the 2 electrodes, such as 75% assigned to electrode 1 and 25% to electrode 2. In another embodiment, an amplitude value is assigned to each of the 2 electrodes, such as 3 mA assigned to electrode 1 and 1 mA to electrode 2. Control of the current in terms of percentages allows precise and consistent distribution of the current between electrodes even as the pulse amplitude is adjusted. It is suited for thinking about the problem as steering a stimulation locus, and stimulation changes on multiple contacts simultaneously to move the locus while holding the stimulation amount constant. Control and displaying the total current through each electrode in terms of absolute values (e.g., mA) allows precise dosing of current through each specific electrode. It is suited for changing the current one contact at a time to shape the stimulation like a piece of clay (pushing/pulling one spot at a time).

Programming control circuit 816 represents an example of programming control circuit 316 and generates the plurality of stimulation parameters, which is to be transmitted to implantable stimulator 704, based on a specified neurostimulation program (e.g., the pattern of neurostimulation pulses as represented by one or more stimulation waveforms and one or more stimulation fields, or at least certain aspects of the pattern). The neurostimulation program may be created and/or adjusted by the user using user interface 810 and stored in external storage device 818. In various embodiments, programming control circuit 816 can check values of the plurality of stimulation parameters against safety rules to limit these values within constraints of the safety rules. In one embodiment, the safety rules are heuristic rules.

User interface 810 represents an example of user interface 310 and allows the user to define the pattern of neurostimulation pulses and perform various other monitoring and programming tasks. User interface 810 includes a display screen 856, a user input device 858, and an interface control circuit 854. Display screen 856 may include any type of interactive or non-interactive screens, and user input device 858 may include any type of user input devices that supports the various functions discussed in this document, such as touchscreen, keyboard, keypad, touchpad, trackball, joystick, and mouse. In one embodiment, user interface 810 includes a GUI. The GUI may also allow the user to perform any functions discussed in this document where graphical presentation and/or editing are suitable as may be appreciated by those skilled in the art.

Interface control circuit 854 controls the operation of user interface 810 including responding to various inputs received by user input device 858 and defining the one or more stimulation waveforms. Interface control circuit 854 includes stimulation programming circuit 320.

In various embodiments, external programming device 802 can have operation modes including a composition mode and a real-time programming mode. Under the composition mode (also known as the pulse pattern composition mode), user interface 810 is activated, while programming control circuit 816 is inactivated. Programming control circuit 816 does not dynamically updates values of the plurality of stimulation parameters in response to any change in the one or more stimulation waveforms. Under the real-time programming mode, both user interface 810 and programming control circuit 816 are activated. Programming control circuit 816 dynamically updates values of the plurality of stimulation parameters in response to changes in the set of one or more stimulation waveforms and transmits the plurality of stimulation parameters with the updated values to implantable stimulator 704.

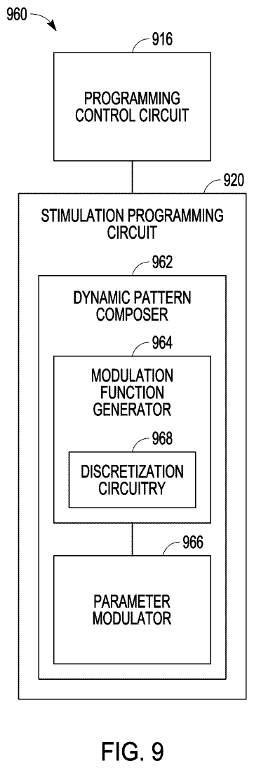

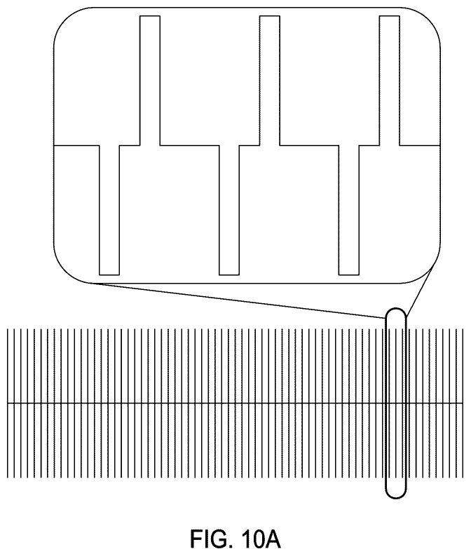

FIG. 9 illustrates an embodiment of a system 960 for programming neurostimulation with one or more dynamic stimulation patterns, including at least one discrete dynamic pattern, to be delivered to a patient. System 960 can include a programming control circuit 916 and a stimulation programming circuit 920. System 960 can be implemented in a neurostimulation system such as systems 100, 500, or 600. In various embodiments, system 960 can be implemented in an external system including one or more programming devices, such as programming device 102, programing device 302, external system 502, CP 630 and RC 632, or external programming device 802. For example, when system 960 is implemented in external programming device 802, programming control circuit 916 is implemented in programming control circuit 816, and stimulation programming circuit 920 is implemented in stimulation programming circuit 320. In various embodiments, system 960 can be implemented in a single device or in two or more devices.

Programming control circuit 920 can generate information for programming a stimulation device to control the delivery of the neurostimulation according to a stimulation program specifying stimulation parameters. The stimulation parameters can include stimulation waveform parameters defining at least one stimulation waveform of the neurostimulation and stimulation field parameters defining at least one stimulation field to which the neurostimulation is to be delivered. Examples of the stimulation device include stimulation device 104, stimulation device 204, IPG 404, IPG 504, IPG 604, and implantable stimulator 704. The stimulation program can include one or more stimulation patterns each defined by the stimulation parameters and a schedule specifying timing for each of the one or more stimulation patterns to be applied to control the delivery of the neurostimulation. The one or more stimulation patterns can include one or more tonic stimulation patterns and/or one or more dynamic stimulation patterns. The one or more dynamic stimulation patterns can include one or more continuous dynamic patterns and/or one or more discrete dynamic patterns.

In various embodiments using one or more dynamic stimulation patterns including at least one discrete dynamic pattern, programming control circuit 916 can generate information for programming the stimulation device to control the delivery of the neurostimulation according to a neurostimulation program including one or more dynamic stimulation patterns each defined by stimulation parameters including a time-varying stimulation parameter. Stimulation programming circuit 920 can determine (e.g., compose) the neurostimulation program and can include a dynamic pattern composer 962. Dynamic pattern composer 962 can determine (e.g., compose) the one or more dynamic stimulation patterns including the discrete dynamic pattern. Dynamic pattern composer 962 can include a modulation function generator 964 and a parameter modulator 966.

To determine a continuous dynamic pattern, modulation function generator 964 can determine a continuous modulation function, which is a continuously time-varying function, and parameter modulator 966 can select a parameter from the stimulation parameters for the continuous dynamic pattern and produce the time-varying stimulation parameter of the continuous dynamic pattern by modulating the selected parameter using the continuous modulation function.

To determine a discrete dynamic pattern, modulation function generator 964 can determine a discretized modulation function, which is a discretely time-varying function. In various embodiments, modulation function generator 964 can receive discretization information and determine the discretized modulation function using the received discretization information. The discretization information can include information that directly identifies a preconfigured discretized modulation function, which can be stored (e.g., in external storage device 818), and modulation function generator 964 can determine the discretized modulation function by selecting the preconfigured discretized modulation function identified by the received discretization information, for example from stored multiple preconfigured discretized modulation functions. Alternatively or additionally, the discretization information can include one or more discretization parameters and/or criteria, and modulation function generator 964 can determine the discretized modulation function by identifying a discretized modulation function, for example from stored multiple preconfigured discretized modulation functions, that matches the one or more discretization parameters and/or criteria. When there is no preconfigured discretized modulation function that can be selected according to the received discretization information, modulation function generator 964 can generate a discretized modulation function using the received discretization information. Modulation function generator 964 can optionally include discretization circuitry 968 (as shown in FIG. 9) that can generate a discretized modulation function using the received discretization information when needed. Discretization circuitry 968 can receive a continuous function and the discretization information and can generate the discretized modulation function by discretizing the continuous function using the discretization information. Parameter modulator 966 can select a parameter from the stimulation parameters for the discrete dynamic pattern and produce the time-varying stimulation parameter of the discrete dynamic pattern by modulating the selected parameter using the discretized modulation function.

When system 960 is implemented in external programming device 802, external storage device 818 can store stimulation programs, stimulation patterns that can each be used in a stimulation program, and/or information for composing stimulation patterns. Such stored information can allow a stimulation program or a stimulation pattern to be determined using predetermined or preconfigured schemes. Presentation device 856 can present each stimulation program and/or each stimulation pattern that are stored in external storage device 818 and/or being composed using stimulation programming circuit 920. User input device 858 can receive information for determining each stimulation program and each stimulation pattern by selecting from the stored stimulation programs and stimulation patterns, respectively, and/or composing a new stimulation programs and a stimulation pattern, respectively.

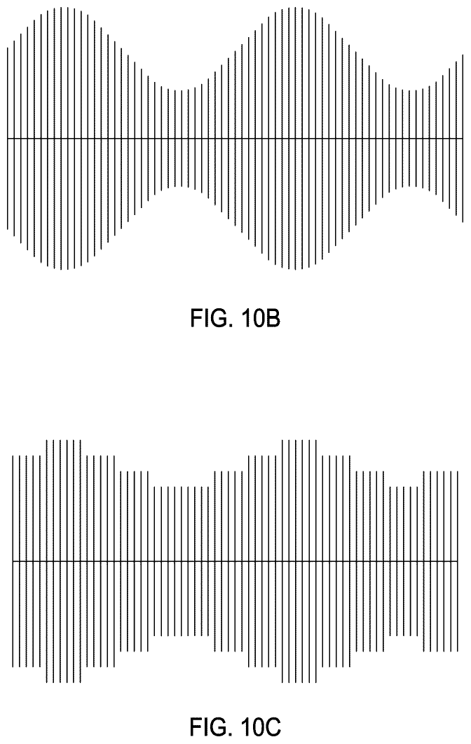

FIGS. 10A-C illustrate examples of stimulation patterns that can be determined using stimulation programming circuit 920, shown for illustrative but not restrictive purposes. FIG. 10A illustrates an example of a tonic stimulation pattern. The illustrated tonic stimulation pattern is a train of pulses defined by time-invariant stimulation parameters including, but not being limited to, a pulse amplitude, a pulse width, a pulse frequency (also referred to as pulse rate), and an interphase interval. Shown in FIG. 10A by way of example but not by way of restriction, the pulses are each a biphasic pulse having a cathodic stimulation phase followed by an anodic recharge phase after the interphase interval. FIG. 10B illustrates an example of a continuous dynamic pattern. The continuous dynamic pattern can result from modulating the pulse amplitude of the tonic stimulation pattern of FIG. 10A with a continuous modulation function. In the case of FIG. 10B, by way of example but not by way of restriction, the continuous modulation function is a continuous sinusoidal function. FIG. 10C illustrates an example of a discrete dynamic pattern. The discrete dynamic pattern can result from modulating the pulse amplitude of the tonic stimulation pattern of FIG. 10A with a discretized modulation function. In the case of FIG. 10C, by way of example but not by way of restriction, the discretized modulation function is a discretized sinusoidal function.

With dynamic stimulation patterns, including discrete dynamic patterns, generated by amplitude modulation (i.e., modulation of the pulse amplitude with a modulation function, including a discretized modulation function) are discussed as specific examples of dynamic stimulation patterns, the present subject matter can also be applied to generate dynamic stimulation patterns, including discrete dynamic patterns, by modulating one or more different stimulation parameters including, but not limited to, the pulse width, the pulse frequency, and/or the interphase interval. Dynamic stimulation patterns, including discrete dynamic patterns, generated according to the present subject matter can include one or more modulated parameters each modulated with an individually selected modulation function using individually specified modulation parameters.

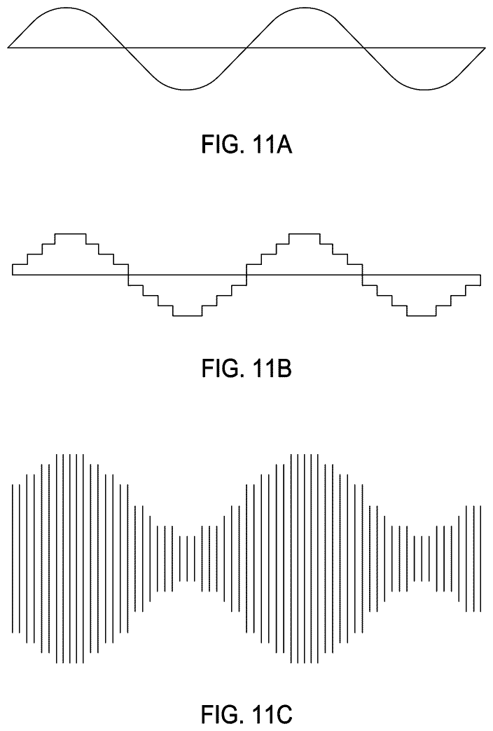

Referring back to FIG. 9, modulation function generator 964 can include discretization circuitry 968. Discretization circuitry 968 can receive a continuous function and discretization information and can generate a discretized modulation function by discretizing the continuous function using the discretization information. FIGS. 11A-C illustrate examples of waveforms in an embodiment of producing a discrete dynamic pattern. FIG. 11A illustrates an example of a continuous function (e.g., a continuous sinusoidal function, as shown). FIG. 11B illustrates an example of a discretized modulation function that can result from discretization of the continuous function of FIG. 11A (e.g., a discretized sinusoidal function, as shown) by discretization circuitry 968. FIG. 11C illustrates an example of a discrete stimulation pattern resulting from modulating the pulse amplitude of a tonic stimulation pattern counterpart using the discretized modulation function of FIG. 11B using parameter modulator 966.

It is to be understood that a “discretized modulation function”, as used herein, refers to a modulation function that can result from discretizing a continuous function rather than digitizing the continuous function (which can be a digitized function already). Accordingly, the discretization information does not include, and differs from, the sampling rate used in the digitization of an analog signal that can be the continuous function. In other words, the term discretization as used herein distinguishes from digitization. The continuous function to be discretized can be an analog function or a digitized function (which is technically a discrete function). The purpose of the discretization according to the present subject matter includes reduction of number of values used in the modulation function. Accordingly, when the (digital) continuous function has a first number of values resulting from digitization at a sampling rate, the discretized modulating function has a second number of values resulting from the discretization of the continuous function, and the second number is smaller than the first number, to an extend determined by the discretization information.

Referring back to FIG. 9, parameters of the modulation function (e.g., type of waveform, frequency, amplitude, modulation depth, and the like) generated using modulation function generator 964 can be empirically determined for a patient population or for an individual patient. In various embodiments, such parameters can be determined using user and/or patient feedback, sensed signals indicative of patient responses to the neurostimulation, and/or results of computer simulations with models of relevant portions of the nervous system.

The discretization information received by discretization circuitry 968 specifies how the continuous function is to be discretized and can include one or more discretization parameters. Using amplitude modulation as an example, the discretization information can include a step number and/or a step size. It is noted that different discretization parameters and/or different values of discretization parameters can be used, for example in different portions of a discretized dynamic pattern (as further discussed below).

The step number can be the number of steps (or increments) in transitioning between a lower amplitude and an upper amplitude (e.g., between positive and negative peaks) of the discretized modulation function (e.g., between positive and negative peaks). The step size can be the magnitude (e.g., amplitude) of each step (or increment) in the discretized modulation function. Alternative and/or additional one or more discretization parameters controlling the magnitude and/or duration of the steps can also be used as understood and determined by those skilled in the art.

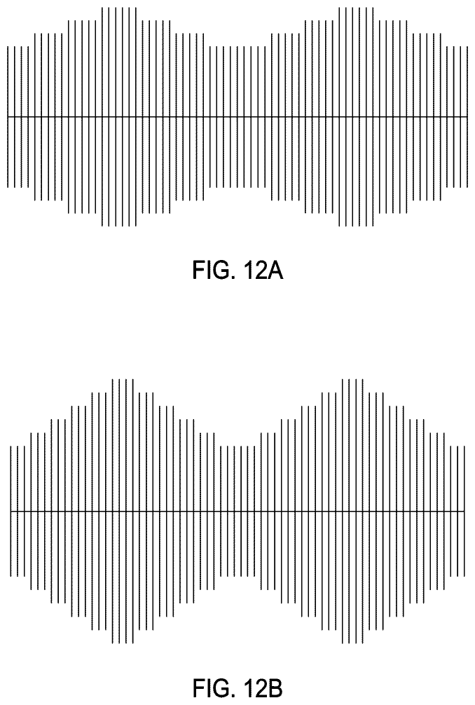

FIGS. 12A-B illustrate examples of discrete dynamic patterns resulting from modulating a pulse amplitude using discretized modulation functions having different step numbers. FIG. 12A illustrates an example of a discrete dynamic pattern resulting from modulating the pulse amplitude using a discretized modulation function having the step number of 4. FIG. 12B illustrates an example of a discrete dynamic pattern resulting from modulating the pulse amplitude using a discretized modulation function having the step number of 6. In various embodiments, the one or more discretization parameters used for generating each discretized modulation function can be empirically determined for a patient population or an individual patient. In various embodiments, the one or more discretization parameters can be determined using user and/or patient feedback, sensed signals indicative of patient responses to the neurostimulation, and/or results of computer simulations with models of relevant portions of the nervous system.

Referring back to FIG. 9, in various embodiments, discretization circuitry 968 can receive the discretization information from a user using a user interface device (e.g., user interface device 810, when the discretization information includes a user-adjustable discretization scheme including the continuous function and/or the one or more discretization parameters) or from a storage device (e.g., external storage device 818, such as when the discretization information identifies preconfigured discretization scheme and/or preconfigured discretized modulation functions.

After the discretized modulation function is determined by modulation function generator 964, parameter modulator 966 can select a parameter from the stimulation parameters of the discrete dynamic pattern to be determined and produce a time-varying stimulation parameter of that discrete dynamic pattern by modulating the selected parameter using the discretized modulation function. While the pulse amplitude is used herein as an example of the selected parameter, other stimulation parameters (e.g., pulse width, pulse frequency, and interphase interval) can also be selected for the modulation. In some embodiments, two or more of the stimulation parameters can be selected for the modulation, simultaneously and/or sequentially. In various embodiments, parameter modulator 966 can receive the selection of the stimulation parameter(s) for the modulation from a user using a user interface device (e.g., user interface device 810, when the user is allowed to make the selection) or from a storage device (e.g., external storage device 818, when the selection is predetermined).

Examples of discretization parameters that can be received and applied by modulation function generator 964 to determine the discretized modulation functions are discussed below. The examples are discussed for illustrative but not restrictive purposes. Unless noted otherwise, modulation of the pulse amplitude is discussed in these examples. However, the concept can be applied to modulation of other stimulation parameters, as understood by those skilled in the art.

Example A. Modulation function generator 964 receives a step number as a discretization parameter and determines the discretized modulation function using the received step number. In one embodiment, discretization circuitry 968 receives the step number and generates the discretized modulation function by discretizing a continuous function using the received step number. The step number corresponds to the number of pulse amplitude values specified in a discrete dynamic pattern. A fixed step number (e.g., range from 1-10) can be specified regardless of the minimum and maximum pulse amplitudes. For example, if the pulse amplitude is to be modulated and ranges from 2.5 mA to 4.0 mA, and the step number is set to 5 (steps), the pulse amplitude will be increased step by step from 2.5 mA to 2.8 mA, 3.1 mA, 3.4 mA, 3.7 mA, and 4.0 mA, i.e., in 5 steps with a step size of 0.3 mA.

Example B. Modulation function generator 964 receives a step size of as a discretization parameter and determines the discretized modulation function using the received step size. In one embodiment, discretization circuitry 968 receives the step size and generates the discretized modulation function by discretizing a continuous function using the received step size. The step size corresponds to the magnitude of each step and is identical for all the steps (e.g., given in milliamperes for modulating a pulse amplitude, microseconds for modulating a pulse width, or pulses per second for modulating a pulse frequency). A fixed step size is not constrained by the corresponding number of steps, while the number of steps is controlled by the step size. For example: if the pulse amplitude is to be modulated and ranges from 2.5 mA to 4.0 mA, and the step size is set to 0.1 (mA/step) the pulse amplitude will be increased from 2.5 mA to 4.0 mA over 15 steps.

Example C. Modulation function generator 964 receives a discretization parameter that is a function of the patient's perception threshold and determines the discretized modulation function using the received discretization parameter. In one embodiment, discretization circuitry 968 receives the discretization parameter being the function of the patient's perception threshold and generates the discretized modulation function by discretizing the continuous function using the received discretization parameter. The perception threshold corresponds to a minimum value of the selected parameter (to be modulated, e.g., the pulse amplitude) above which the patient perceives the neurostimulation being delivered. A step size can be set based on patient perception, either relative to the stimulation parameter (e.g., the pulse amplitude) or to an absolute value, as determined or calibrated based on a patient's perception threshold (e.g., a minimum pulse amplitude at which the patient can feel the neurostimulation being delivered). Variations in the perception threshold between the patient's postures (e.g. seated vs. supine), and/or variations of a discomfort threshold, can be factors used in setting the step size. The discomfort threshold corresponds to a maximum value of the selected parameter above which the patient perceives the neurostimulation as being uncomfortable, unacceptable, or intolerable. For example, a patient whose perception to discomfort threshold range covers 2.0 mA may have a program configured with a larger step size than a patient whose perception to discomfort threshold range covers 1.0 mA. In another example, a discrete dynamic pattern may be configured to increase the step size when a posture sensor (e.g., an accelerometer) detects that the patient is seated and to decrease to step size when the posture sensor detects a shift to a reclining or supine posture.

Example D. Modulation function generator 964 receives a discretization parameter (e.g., step size) that has multiple values corresponding to ranges of the selected parameter and determines the discretized modulation function using the received discretization parameter. In one embodiment, discretization circuitry 968 receives the discretization parameter having multiple values corresponding to ranges of the selected stimulation parameter (to be modulated) and generates the discretized modulation function by discretizing the continuous function using the discretization parameter. The patient may be more sensitive to zones or ranges of the selected stimulation parameter, and the step size can be set for each zone or range independently. In an example of pulse width modulation (the selected stimulation parameter is the pulse width) with the pulse width varying between 100 μs and 300 μs, a patient has a discomfort threshold with a 90-Hz, 2.0-mA pulse train when the pulse width is between 200 μs and 300 μs and feel discomfort when the discomfort threshold is exceeded, a discrete dynamic pattern can include coarse steps for the pulse width between 100 μs and 200 μs (e.g., 2 steps, and the neurostimulation is sub-perceptive) and fine steps for the pulse width between 200 μs and 300 μs (e.g., 8 steps). In the case of pulse width modulation (the selected stimulation parameter is the pulse width), the step size may also be approximated using strength-duration relationships, e.g., by using the pulse width difference required to change a threshold by a fixed factor. In this case, pulse widths above a reference value, e.g., chronaxie, may be varied with larger step sizes, and pulse widths below chronaxie may correspondingly be varied with smaller step sizes.

Example E. Modulation function generator 964 determines a discretization parameter (e.g., step size) based on settings of one or more stimulation parameters of the stimulation parameters other than the selected parameter and determines the discretized modulation function using the determined discretization parameter. In one embodiment, discretization circuitry 968 determines the discretization parameter based on settings of one or more stimulation parameters other than the selected parameter and generates the discretized modulation function by discretizing the continuous function using the determined discretization parameter. Using a known relationship between the stimulation parameters, step sizes for a stimulation parameter can be recommended based on settings of the other stimulation parameter(s). For example, using the strength-duration relationship, step sizes for one of the pulse amplitude and the pulse width can be recommended based on a setting of the other one of the pulse amplitude and the pulse width. For example, a step size of 0.3 mA can be recommended for the pulse amplitude when the pulse width is set to 200 μs for a patient with a high perception threshold, whereas in that same patient, a step size of 0.1 mA can be recommended for the pulse amplitude when the pulse width is set to 350 μs due to the longer pulse width lowering activation thresholds. Such known relationships between stimulation parameters can be deterministic e.g., (based on strength-duration) or empirical (e.g., if different stimulation fields are also considered).

Example F. Modulation function generator 964 determines a discretization parameter (e.g., step size) based on a signal sensed from the patient and determines the discretized modulation function using the determined discretization parameter. In one embodiment, discretization circuitry 968 determines the discretization parameter based on a sensed signal and generates the discretized modulation function by discretizing the continuous function using the determined discretization parameter. The discretization parameter (e.g., step size) can be can be determined and/or adjusted based on the magnitude (e.g., change or progression of the magnitude) of sensed evoked potentials. The evoked potentials can each be evoked by a neurostimulation pulse. An example of such evoked potentials include evoked compound action potentials (ECAPs), which can be detected from, for example, electrospinogram (ESG). For example, a step size can be set to decrease when the rate at which an ECAP amplitude increases as a stimulation parameter, such as the pulse amplitude or the pulse width, is increased. Alternatively, the step size can be set to decrease as the ECAP amplitude approaches a calibrated value associated with patient discomfort (or to increase as the ECAP amplitude falls below that value, depending on other available information, such as postural changes and sensing location consistency).

Example G. Modulation function generator 964 determines a discretization parameter (e.g., step size) based on an amount of charge delivered to the patient by the delivery of the neurostimulation and determines the discretized modulation function using the determined discretization parameter. In one embodiment, discretization circuitry 968 determines the discretization parameter based on an amount of charge delivered to the patient by the delivery of the neurostimulation and generates the discretized modulation function by discretizing the continuous function using the determined discretization parameter. The discretization parameter can be set based on charge delivered rather than a single stimulation parameter. The step size can be set according to the total charge delivered over a period of time and/or the charge delivered per pulse, rather than according to a single stimulation parameter such as the pulse amplitude, the pulse width, or the pulse frequency. For example, the “dose” (when measured by the charge delivered to the patient) can be set to vary between 200 nC/pulse and 400 nC/pulse in 50 nC increments. The pulse amplitude and the pulse width are set for delivering that dose, or charge, while their specific values are unconstrained. Values of the pulse amplitude and the pulse width that provide for the same level of charge can vary pulse by pulse within the discrete dynamic pattern.

Examples A-G above are discussed for illustrating specific functions that can be performed using modulation function generator 964 by way of example, but not by way of restriction. In some embodiments, discretization schemes discussed in two or more of these examples can be applied in determining a discrete dynamic pattern and/or a neurostimulation program including multiple discrete dynamic patterns.

In various embodiments, dynamic pattern composer 962 can compose a dynamic stimulation pattern toggling between a continuous dynamic pattern and a discrete dynamic pattern. Similarly, dynamic pattern composer 962 can compose a dynamic stimulation pattern toggling between a discrete dynamic pattern with a large step size and another discrete dynamic pattern with a small step size. For example, a dynamic stimulation pattern can include a “low dose” portion and a “high dose” portion. During the low dose portion, a discrete dynamic pattern with coarse steps can be applied. During the high dose portion, a continuous dynamic pattern or a discrete dynamic pattern with fine steps can be applied.

In various embodiments, stimulation programming circuit 920 can determine a neurostimulation program including one or more tonic stimulation patterns and/or one or more dynamic stimulation patterns. The one or more dynamic stimulation patterns can include one or more discrete dynamic patterns and/or one or more continuous dynamic patterns. The dynamic stimulation pattern is a discrete dynamic pattern when at least one modulated stimulation parameter is modulated by a discretized modulation function. The dynamic stimulation pattern is a continuous dynamic pattern when any modulated stimulation parameter is modulated by a continuous modulation function. In various embodiments, stimulation programming circuit 920 can determine a neurostimulation program including multiple dynamic stimulation patterns and a schedule specifying a time period for each dynamic stimulation pattern of the multiple dynamic stimulation patterns to be applied for controlling the delivery of the neurostimulation.

In one embodiment, one or more discrete dynamic patters are incorporated into an “auto-dose” algorithm which automatically adjusts the charges delivered with the neurostimulation to the patient, for example, over different periods of time. For example, a neurostimulation program specifies repeated cycles of a long period of low dose followed by a short period of high dose. A different stimulation pattern can be applied in each of these periods, with the different stimulation pattern selected from tonic stimulation patterns, continuous stimulation pattern, and discrete stimulation patterns.

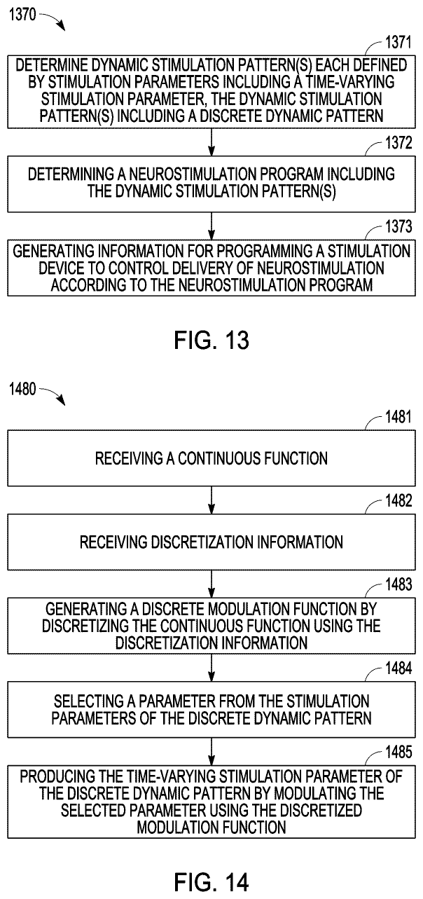

FIG. 13 illustrates an embodiment of a method 1370 for programming a stimulation device for delivering neurostimulation to a patient according to one or more dynamic stimulation patterns including a discrete dynamic pattern. Examples of the stimulation device include stimulation device 104 and its various embodiments as discussed herein (e.g., stimulation device 204, IPG 404, IPG 504, IPG 604, and implantable stimulator 704). System 960 can be configured for performing method 1370. A non-transitory computer-readable storage medium can include instructions, which when executed by a system, such as system 960, cause the system to perform method 1370. For example, when system 960 is implemented in external programming device 802, the instructions can be stored in external storage device 818, to be executed by external programming device 802 using a processor of interface control circuit 854.

At 1371, one or more dynamic stimulation patterns including at least one discrete dynamic pattern are determined. The one or more dynamic stimulation patterns are each defined by stimulation parameters including a time-varying stimulation parameter. The one or more dynamic stimulation patterns can be multiple dynamic stimulation patterns that also include a continuous dynamic pattern. The continuous dynamic pattern can be determined by determining a continuous modulation function, selecting a parameter from the stimulation parameters of the continuous dynamic pattern, and producing the time-varying stimulation parameter of the continuous dynamic pattern by modulating the selected parameter using the continuous modulation function. In various embodiments, the continuous dynamic pattern can include one or more time-varying stimulation parameters. The continuous modulation function can be individually selected to modulate each time-varying stimulation parameter. The discrete dynamic pattern can be determined by determining a discretized modulation function, selecting a parameter from the stimulation parameters of the discrete dynamic pattern, and producing the time-varying stimulation parameter of the discrete dynamic pattern by modulating the selected parameter using the discretized modulation function. In various embodiments, the discrete dynamic pattern can include one or more time-varying stimulation parameters. The discrete modulation function can be individually selected to modulate each time-varying stimulation parameter. In various embodiments, the discrete dynamic pattern can be determined using a preconfigured discretized modulation function or a discretized modulation function generated using a continuous function and discretization information. An example of determination of the discrete dynamic pattern using a continuous function and discretization information is discussed below with reference to FIG. 14.

At 1372 a neurostimulation program including the one or more dynamic stimulation patterns is determined. The neurostimulation program can include the discrete dynamic pattern and the continuous dynamic pattern. The determination of the neurostimulation program can also include scheduling time periods for the discrete dynamic pattern and the continuous dynamic pattern such that the discrete dynamic pattern and the continuous dynamic pattern are applied at different times.

At 1373, information for programming the stimulation device to control the delivery of the neurostimulation according to the neurostimulation program is generated. The stimulation device can be programmed accordingly, to deliver the neurostimulation to the patient and control the delivery of the neurostimulation according to the stimulation program

FIG. 14 illustrates an embodiment of a method 1480 for determining the discrete dynamic pattern in method 1370. System 960 (particularly dynamic pattern composer 642) can be configured for performing method 1480. A non-transitory computer-readable storage medium can include instructions, which when executed by a system, such as system 960, cause the system to perform method 1480. For example, when system 960 is implemented in external programming device 802, the instructions can be stored in external storage device 818, to be executed by external programming device 802 using a processor of interface control circuit 854.

At 1481, a continuous function is received. At 1482, discretization information is received. The discretization information can include one or more discretization parameters including a step number and/or a step size. The step number defines a number of steps in transitioning between a lower amplitude and an upper amplitude of the continuous function. The step size defines a magnitude of each step of the steps. In various embodiments, the one or more discretization parameters can be determined as a function of the patient's perception threshold. At 1483, a discretized modulation function is generated by discretizing the continuous function using the discretization information. At 1484, a parameter is selected from the stimulation parameters of the discrete dynamic pattern. At 1485, the time-varying stimulation parameter of the discrete dynamic pattern is produced by modulating the selected parameter using the discretized modulation function.

In various embodiments, the discrete dynamic pattern can include one or more time-varying stimulation parameters. Steps 1481, 1482, 1483, 1484, and 1485 can be repeated for each time-varying stimulation parameter when the stimulation parameters include more than one time-varying stimulation parameter. The continuous function and the discretization parameter can be individually determined for each time-varying stimulation parameter.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for delivering neurostimulation from a stimulation device to a patient, the system comprising:
a programming control circuit configured to generate information for programming the stimulation device to control the delivery of the neurostimulation according to a neurostimulation program including one or more dynamic stimulation patterns each defined by stimulation parameters including at least one time-varying stimulation parameter; and
a stimulation programming circuit configured to determine the neurostimulation program, the stimulation programming circuit including a dynamic pattern composer configured to determine a discrete dynamic pattern of the one or more dynamic stimulation patterns, the dynamic pattern composer including:
a modulation function generator configured to receive discretization information and to determine a discretized modulation function using the received discretization information, the discretized modulation function being a discretely time-varying function, and
a parameter modulator configured to select a parameter from the stimulation parameters defining the discrete dynamic pattern and to produce the at least one time-varying stimulation parameter of the discrete dynamic pattern by modulating the selected parameter using the discretized modulation function.

2. The system of claim 1, wherein the modulation function generator is configured to receive one or more discretization parameters of the discretization information and to determine the discretized modulation function according to the one or more discretization parameters.

3. The system of claim 2, wherein the modulation function generator comprises discretization circuitry configured to receive a continuous function, to receive the one or more discretization parameters, and to generate the discretized modulation function by discretizing the continuous function using the one or more discretization parameters.

4. The system of claim 2, wherein the modulation function generator is configured to receive at least one of a step number or a step size of the one or more discretization parameters and to determine the discretized modulation function using the received at least one of the step number or the step size, the step number defining a number of steps in transitioning between a lower amplitude and an upper amplitude specified for the discretized modulation function, the step size defining a magnitude of each step of the steps in transitioning between the lower amplitude and the upper amplitude specified for the discretized modulation function.

5. The system of claim 2, wherein the modulation function generator is configured to receive a discretization parameter of the one or more discretization parameters that is a function of the patient's perception threshold and to determine the discretized modulation function using the received discretization parameter, the perception threshold corresponding to a minimum value of the selected parameter for the patient to perceive the neurostimulation being delivered.

6. The system of claim 2, wherein the modulation function generator is configured to receive a discretization parameter of the one or more discretization parameters that has multiple values corresponding to ranges of the selected parameter and to determine the discretized modulation function using the received discretization parameter.

7. The system of claim 2, wherein the modulation function generator is configured to determine a discretization parameter of the one or more discretization parameters based on settings of one or more stimulation parameters of the stimulation parameters other than the selected parameter and to determine the discretized modulation function using the determined discretization parameter.

8. The system of claim 2, wherein the modulation function generator is configured to determine a discretization parameter of the one or more discretization parameters based on a signal sensed from the patient and to determine the discretized modulation function using the determined discretization parameter.

9. The system of claim 2, wherein the modulation function generator is configured to determine a discretization parameter of the one or more discretization parameters based on an amount of charge delivered to the patient by the delivery of the neurostimulation and to determine the discretized modulation function using the determined discretization parameter.

10. The system of claim 1, wherein the stimulation programming circuit is configured to determine a neurostimulation program including multiple dynamic stimulation patterns and a schedule specifying a time period for each dynamic stimulation pattern of the multiple dynamic stimulation patterns to be applied for controlling the delivery of the neurostimulation, and the dynamic pattern composer is configured to determine a continuous dynamic pattern of the multiple dynamic stimulation patterns, the modulation function generator is configured to generate a continuous modulation function that is a continuously time-varying function, and the parameter modulator is configured to select a parameter from the stimulation parameters of the continuous dynamic pattern and to produce the at least one time-varying stimulation parameter of the continuous dynamic pattern by modulating the selected parameter using the continuous modulation function.

11. A method for delivering neurostimulation from a stimulation device to a patient, the method comprising:
determining one or more dynamic stimulation patterns each defined by stimulation parameters including at least one time-varying stimulation parameter, the one or more dynamic stimulation patterns including a discrete dynamic pattern, the determination of the discrete dynamic pattern including:
receiving discretization information;
determining a discretized modulation function using the received discretization information, the discretized modulation function being a discretely time-varying function;
selecting a parameter from the stimulation parameters of the discrete dynamic pattern; and
producing the at least one time-varying stimulation parameter of the discrete dynamic pattern by modulating the selected parameter using the discretized modulation function;
determining a neurostimulation program including the one or more dynamic stimulation patterns; and
generating information for programming the stimulation device to control the delivery of the neurostimulation according to the neurostimulation program.

12. The method of claim 11, wherein receiving the discretization information comprises receiving one or more discretization parameters including at least one of a step number or a step size, the step number defining a number of steps in transitioning between a lower amplitude and an upper amplitude specified for the discretized modulation function, the step size defining a magnitude of each step of the steps.

13. The method of claim 12, wherein receiving the one or more discretization parameters comprises receiving a discretization parameter being a function of the patient's perception threshold, the perception threshold corresponding to a maximum value of the selected parameter tolerable by the patient.

14. The method of claim 12, wherein receiving the one or more discretization parameters comprises receiving a discretization parameter having multiple values corresponding to ranges of the selected parameter.

15. The method of claim 12, wherein the determination of the discrete dynamic pattern further comprises determining a discretization parameter of the one or more discretization parameters based on settings of one or more stimulation parameters of the stimulation parameters other than the selected parameter.

16. The method of claim 12, wherein the determination of the discrete dynamic pattern further comprises determining a discretization parameter of the one or more discretization parameters based on a signal sensed from the patient.

17. The method of claim 12, wherein the determination of the discrete dynamic pattern further comprises determining a discretization parameter of the one or more discretization parameters based on an amount of charge delivered to the patient by the delivery of the neurostimulation.

18. The method of claim 11, wherein determining the neurostimulation program comprises determining a neurostimulation program including multiple dynamic stimulation patterns including the discrete dynamic pattern and a continuous dynamic pattern, and determining the one or more dynamic stimulation patterns comprises determining the continuous dynamic pattern, including:
generating a continuous modulation function that is a continuously time-varying function;
selecting a parameter from the stimulation parameters of the continuous dynamic pattern; and
producing the at least one time-varying stimulation parameter of the continuous dynamic pattern by modulating the selected parameter using the continuous modulation function.

19. The method of claim 18, wherein determining the neurostimulation program comprises scheduling time periods for the discrete dynamic pattern and the continuous dynamic pattern such that the discrete dynamic pattern and the continuous dynamic pattern are applied at different times.

20. A non-transitory computer-readable storage medium including instructions, which when executed by a system, cause the system to perform a method for delivering neurostimulation from a stimulation device to a patient, the method comprising:
determining one or more dynamic stimulation patterns each defined by stimulation parameters including at least one time-varying stimulation parameter, the one or more dynamic stimulation patterns including a discrete dynamic pattern, the determination of the discrete dynamic pattern including:
receiving discretization information;
determining a discretized modulation function using the received discretization information, the discretized modulation function being a discretely time-varying function;
selecting a parameter from the stimulation parameters of the discrete dynamic pattern; and
producing the at least one time-varying stimulation parameter of the discrete dynamic pattern by modulating the selected parameter using the discretized modulation function;
determining a neurostimulation program including the one or more dynamic stimulation patterns; and
generating information for programming the stimulation device to control the delivery of the neurostimulation according to the neurostimulation program.
